(19) United States
(12) Patent Application Publication
(10) Pub. No.: US 2013/0172946 A1
(43) Pub. Date: Jul. 4, 2013

(54) IMPLANT MODULE AND METHOD FOR REPAIRING AVULSION FRACTURE

(71) Applicants: Ching-Chuan Jiang, New Taipei City (TW); Chun-Jen Liao, Taipei City (TW); Wen-Hsiang Chang, Hsinchu City (TW); Shih-Jui Han, Taichung City (TW); Yu-Ming Wang, Tainan City (TW); Ying-Wen Shen, Miaoli County (TW)

(72) Inventors: Ching-Chuan Jiang, New Taipei City (TW); Chun-Jen Liao, Taipei City (TW); Wen-Hsiang Chang, Hsinchu City (TW); Shih-Jui Han, Taichung City (TW); Yu-Ming Wang, Tainan City (TW); Ying-Wen Shen, Miaoli County (TW)

(73) Assignee: INDUSTRIAL TECHNOLOGY RESEARCH INSTITUTE, Hsinchu (TW)

(21) Appl. No.: 13/565,822

(22) Filed: Aug. 3, 2012

(30) Foreign Application Priority Data
Dec. 28, 2011 (TW) 100149291

(51) Int. Cl. A61B 17/86; A61B 17/88

(52) U.S. Cl. 606/301

(57) ABSTRACT

An implant module for dragging a bone fragment toward a bone is provided. The bone fragment has a first through hole, and the bone has a second through hole. The implant module includes a screw and a stud. The screw has a first section fixed to the bone fragment and a second section provided with a first external thread on its outer surface. The stud is provided with a second external thread on its outer surface for being screwed into the second through hole of the bone. An end of the stud has a screw hole for being screwed with the second section of the screw via the first external thread. A method of repairing avulsion fracture by using the implant module is further provided.

### CROSS REFERENCE TO RELATED APPLICATIONS

This application claims the priority benefit of Taiwan application serial no. 100149291, filed on Dec. 28, 2011. The entirety of the above-mentioned patent application is hereby incorporated by reference herein and made a part of this specification.

### TECHNICAL FIELD

The disclosure relates to an implant module and a method for repairing an avulsion fracture by applying the same.

### BACKGROUND

Being the toughest ligament in the knee, the posterior cruciate ligament (PCL) has a main function of preventing posterior translation of tibia. For example, the posterior cruciate ligament provides about 85% to 100% of a force for resisting the posterior translation of tibia when the knee is bent to 30 degrees or 90 degrees.

When the knee sustains a serious wound, such as car accidents, falls from height, or severe sports injuries, etc., the external force causing pivot or excessive inflection of the knee may exceed the tension which the posterior cruciate ligament can withstand, and thereby leads to partial damage, complete rupture or avulsion fracture of the posterior cruciate ligament. The avulsion fracture occurs when the posterior cruciate ligament together with a bone fragment at an end thereof are torn from a bone.

FIG. 1A and FIG. 1B are respectively different views showing an avulsion fracture of a posterior cruciate ligament behind and in front of a knee.

Recently, invasive surgical treatments with large wounds are adopted to repair the avulsion fracture of the posterior cruciate ligament.

Therefore, postoperative patients with incision need the joint brackets to protect the knee for about three months, use crutches for about one to two months, walk ordinarily after three months, and are capable of exercise after six months, which cause inconvenience to the postoperative patients in daily life.

### SUMMARY

The present application provides an implant module for dragging a bone fragment toward where the bone fragment tears from a bone. The bone fragment has a first through hole, and the bone has a second through hole penetrating the bone and being connected to where the bone fragment tears from the bone. The implant module comprises a screw and a stud. The screw including a first section and a second section, wherein the first section is fixed to the bone fragment, and the second section is provided with a first external thread on its outer surface. The stud is provided with a second external thread on its outer surface for being screwed with the second through hole of the bone, wherein an end of the stud has a screw hole, and the second section of the screw is adapted to be screwed with the screw hole via the first external thread.

One embodiment provides an implant module including a screw and a stud. The screw comprises a first section and a second section. The second section has a first external thread on its outer surface. The stud is provided with a second external thread on its outer surface, and an end of the stud has a screw hole adapted to screw with the second section of the screw via the first external thread.

Another embodiment provides a method for repairing an avulsion fracture to drag a bone fragment toward where the bone fragment tears from a bone. The method comprising: forming a first through hole in the bone fragment, and forming a second through hole in the bone, wherein the second through hole penetrates the bone and is connected to where the bone fragment tears from the bone; screwing a screw to the bone fragment from a side adjacent to where the bone fragment tears from the bone, wherein a first section of the screw is fixed to the bone fragment; and, screwing a stud to the bone from a side far away from where the bone fragment tears from the bone, wherein the stud with a second external thread on an outer surface thereof is screwed with the second through hole of the bone, and an end of the stud has a screw hole screwed with a first external thread on a second section of the screw, to drag the bone fragment toward where the bone fragment tears from the bone.

As to the above, the implant module and the method for repairing the avulsion fracture are provided with a stud having a second external thread in a pitch different from a pitch of a first external thread of a screw, wherein the screw fixed to the bone fragment can be dragged toward the stub in the bone by screwing the stud with the screw, so as to repair the avulsion fracture. Since the implant module is in a small size, small incisions of surgical treatment are formed for implanting the implanting module, and thereby the recovery time of patients can be reduced.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1A and FIG. 1B are respectively different views showing an avulsion fracture of a posterior cruciate ligament behind and in front of a knee.

FIG. 2A is an explosive view of an implant module according to an embodiment of the disclosure.

FIG. 2B shows a cap and a screw of the implant module of FIG. 2A being assembled.

FIG. 3A through FIG. 3L shows the method for repairing the avulsion fracture according to an embodiment of the disclosure.

FIG. 4A through FIG. 4E are partial enlarged views showing the structures in FIG. 3H through FIG. 3L in another view.

FIG. 5A is an explosive view of an implant module according another embodiment of the disclosure.

FIG. 5B shows a cap and a screw of the implant module of FIG. 5A being assembled.

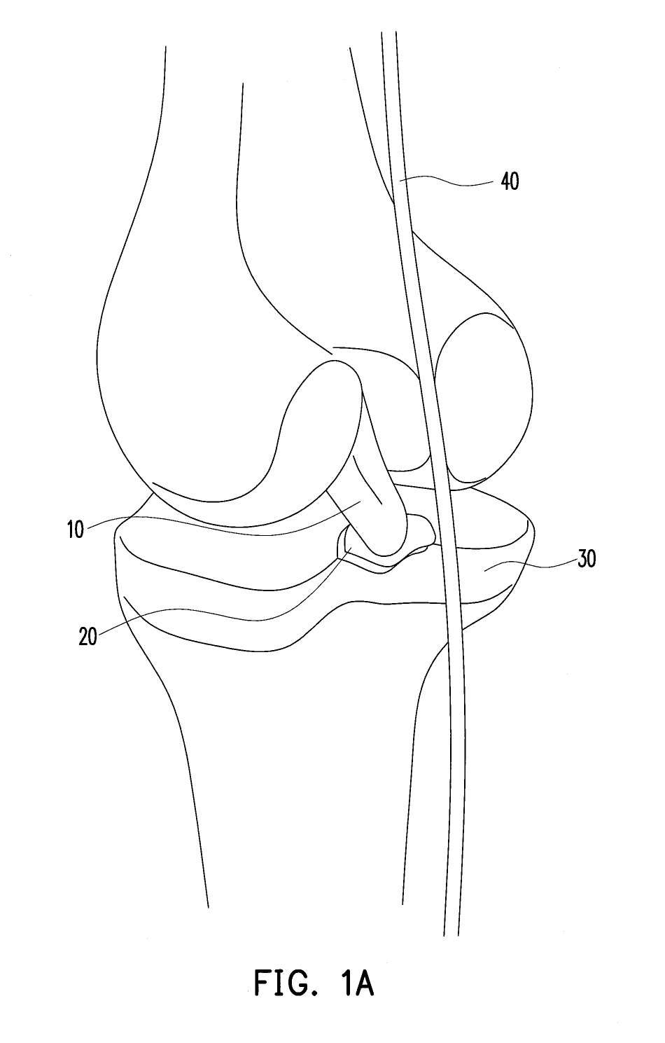

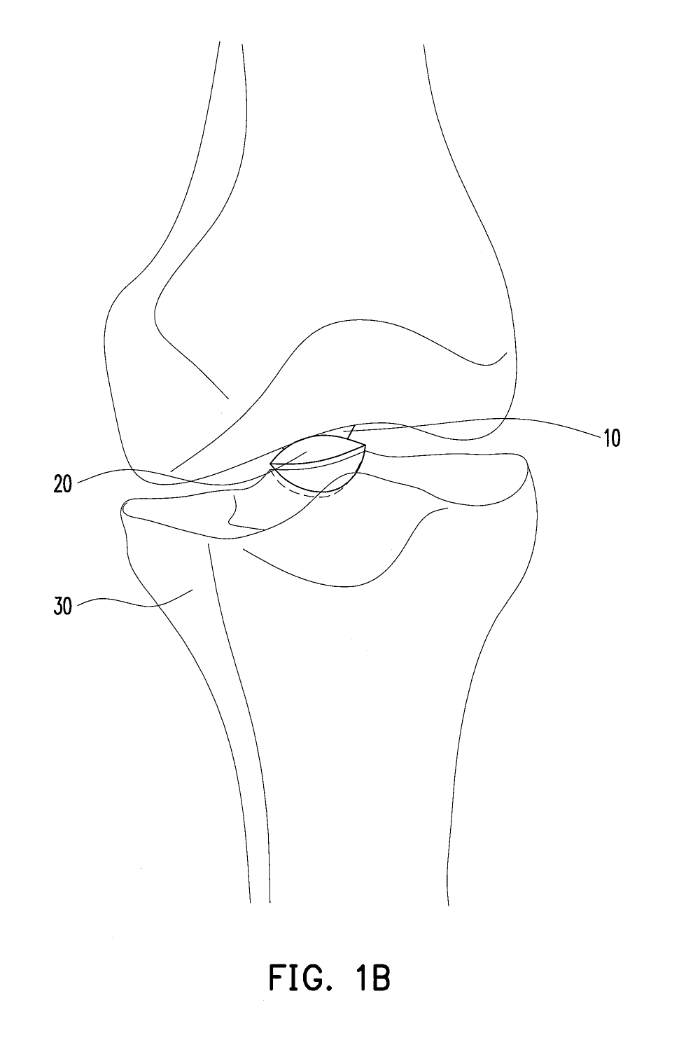

### DETAILED DESCRIPTION OF DISCLOSED EMBODIMENTS

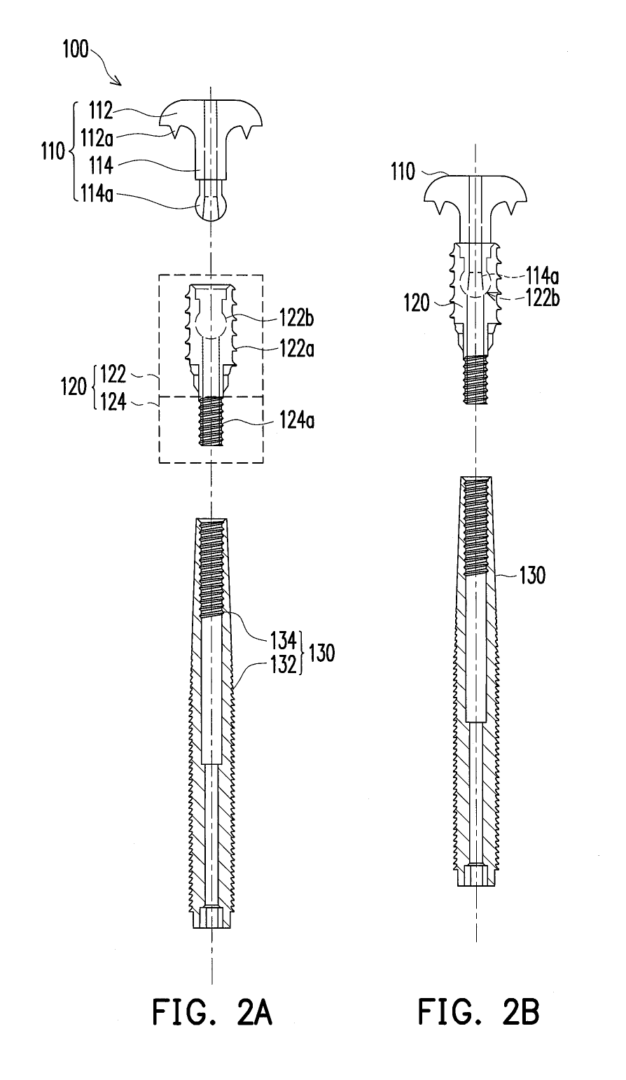

An implant module is provided to drag a bone fragment toward where the bone fragment tears from a bone. FIG. 2A is an explosive view of an implant module according to an embodiment of the disclosure. FIG. 2B shows a cap and a screw of the implant module of FIG. 2A being assembled. Referring to FIG. 2A and FIG. 2B, the implant module 100 comprises the cap 110, the screw 120 and a stud 130.

The cap 110 comprises a head portion 112 and a stem portion 114 connected to the head portion 112. The stem portion 114 is adapted to penetrate the bone fragment (not shown) from a side far away from the bone (not shown), to prop the head portion 112 against the side of the bone fragment far away from the bone. An end of the stem portion 114 penetrating the bone fragment has a first engaging portion 114a.

The screw 120 comprises a first section 122 and a second section 124. The first section 122 has a third external thread 122a on its outer surface to be screwed with the bone fragment. The second section 124 has first external thread 124a on its outer surface. The first section 122 has a second engaging portion 122b adapted to be engaged with the first engaging portion 114a, to link the cap 110 and the screw 120.

The stud 130 has a second external thread 132 on its outer surface, to be screwed with the bone. An end of the stud 130 has a screw hole 134, and the first external thread 124a is matched with the screw hole 134, such that the second section 124 of the screw 120 can be screwed with the screw hole 134 via the first external thread 124a.

A pitch of the second external thread 132 is configured to be different from a pitch of the first external thread 124a such that the stud 130 moves with respective to the bone in a distance smaller than a displacement between the screw 120 and the stub 130 when the stud 130 is screwed with the bone via the second external thread 132 and screwed with the first external thread 124a of the screw 120 via the screw hole 134, for dragging the bone fragment toward where the bone fragment tears from the bone and resetting the bone fragment to its original location. The bone fragment is clamped by the cap 110 and the screw 120, wherein the cap 110 and the screw 120 are fixed with each other such that the bone fragment moves toward stud 130 with the screw 120 and is dragged toward where the bone fragment tears from the bone. In the present embodiment, the screw 120 is reliably fixed to the bone fragment by using the cap 110. However, the cap 110 can further be replaced by using other manners such as screwing, adhering, and etc., to fixing the screw 120 to the bone fragment.

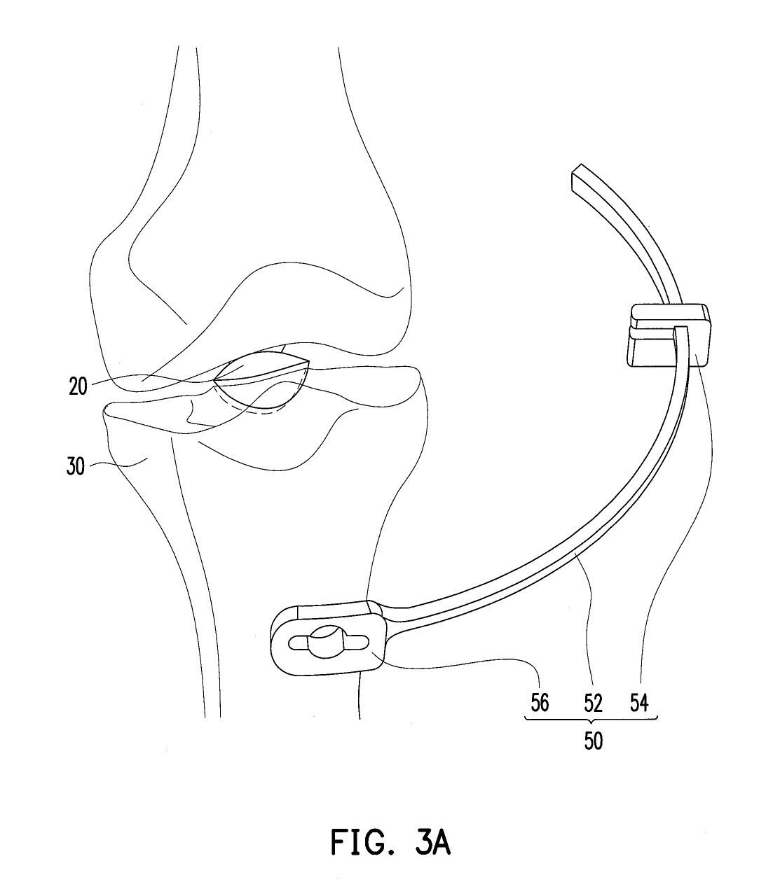

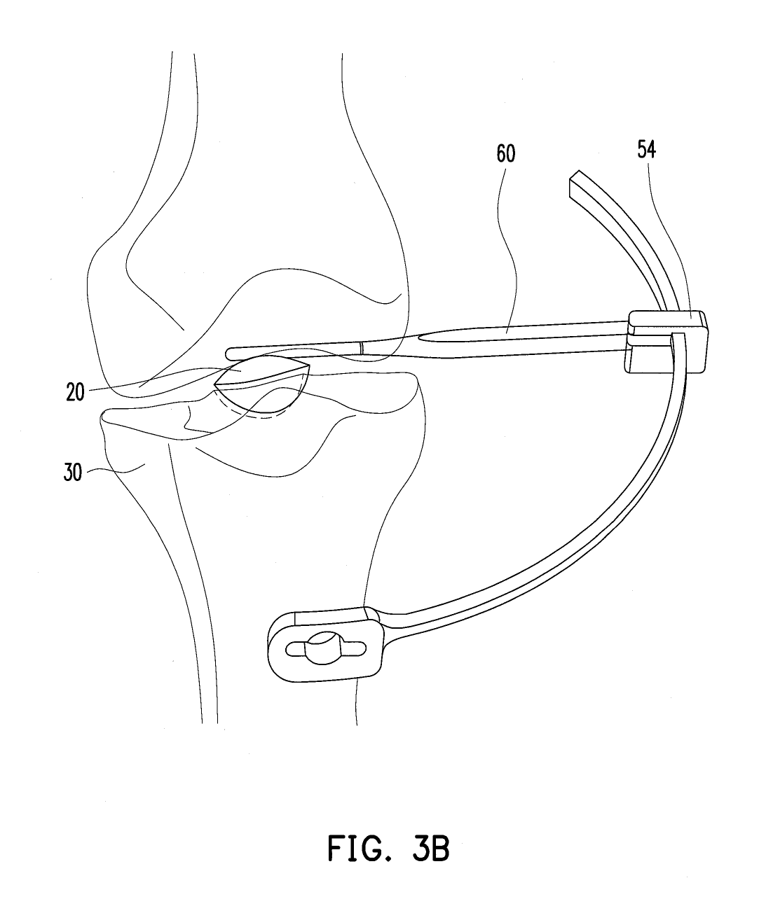

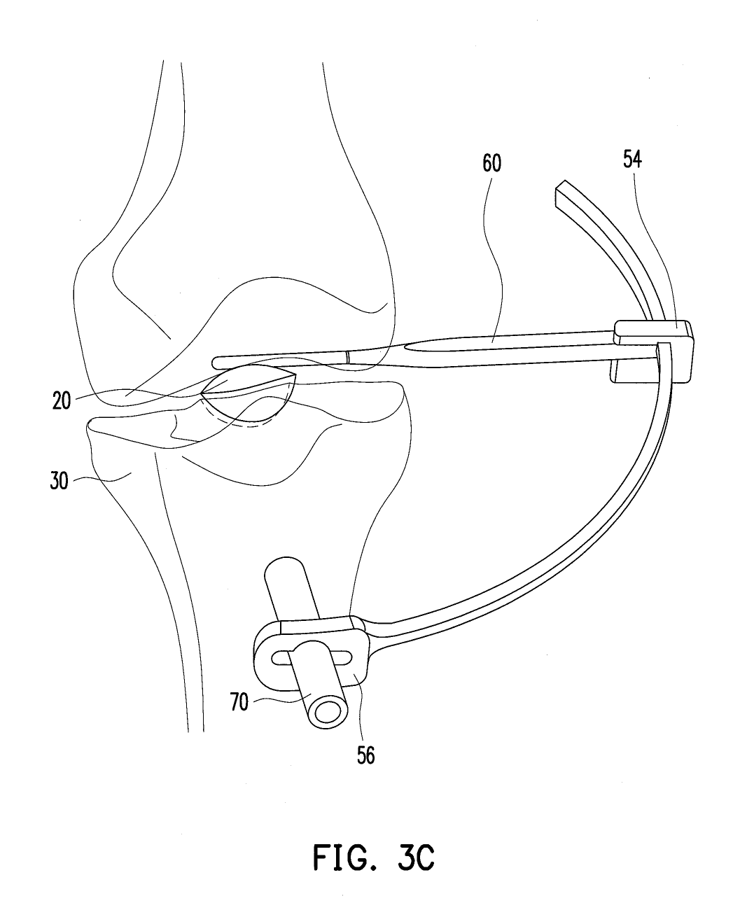

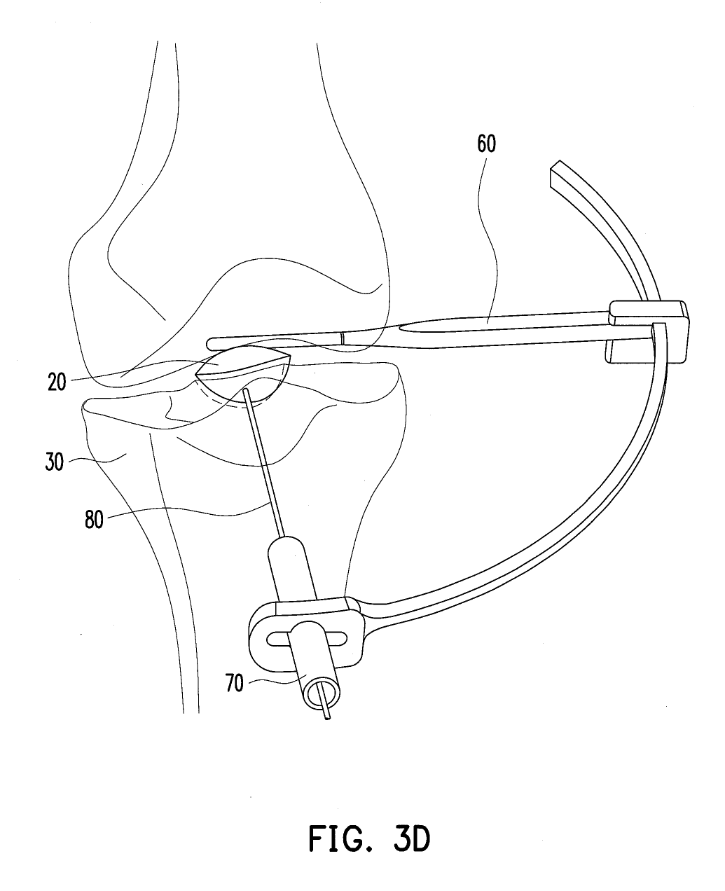

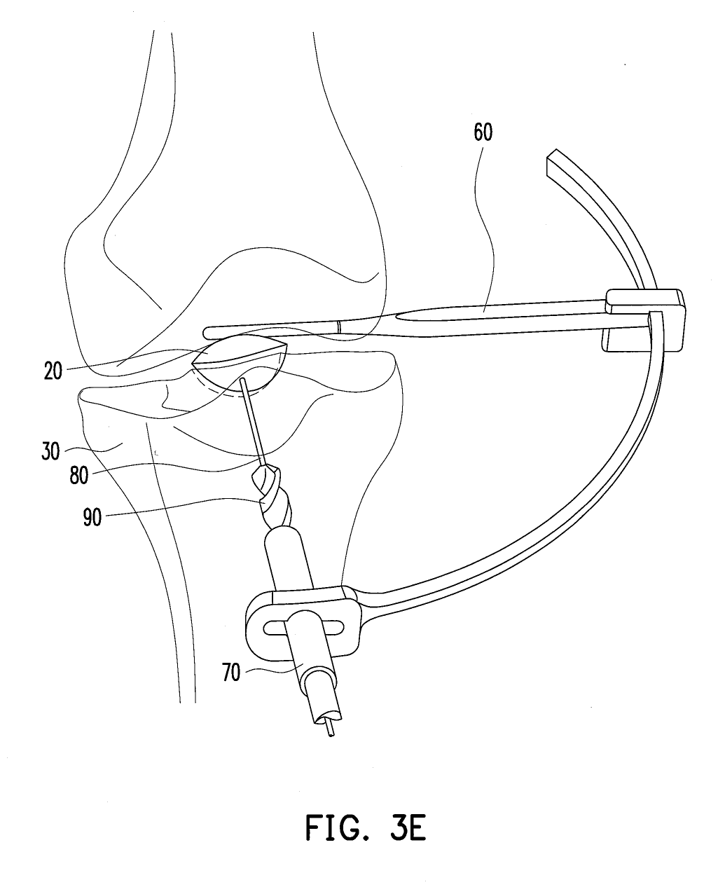

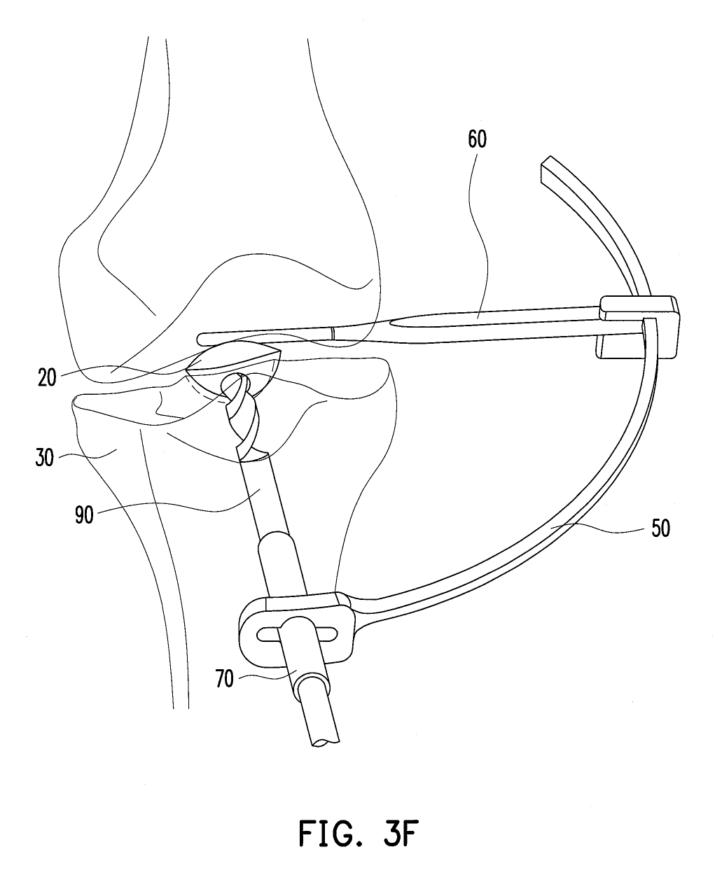

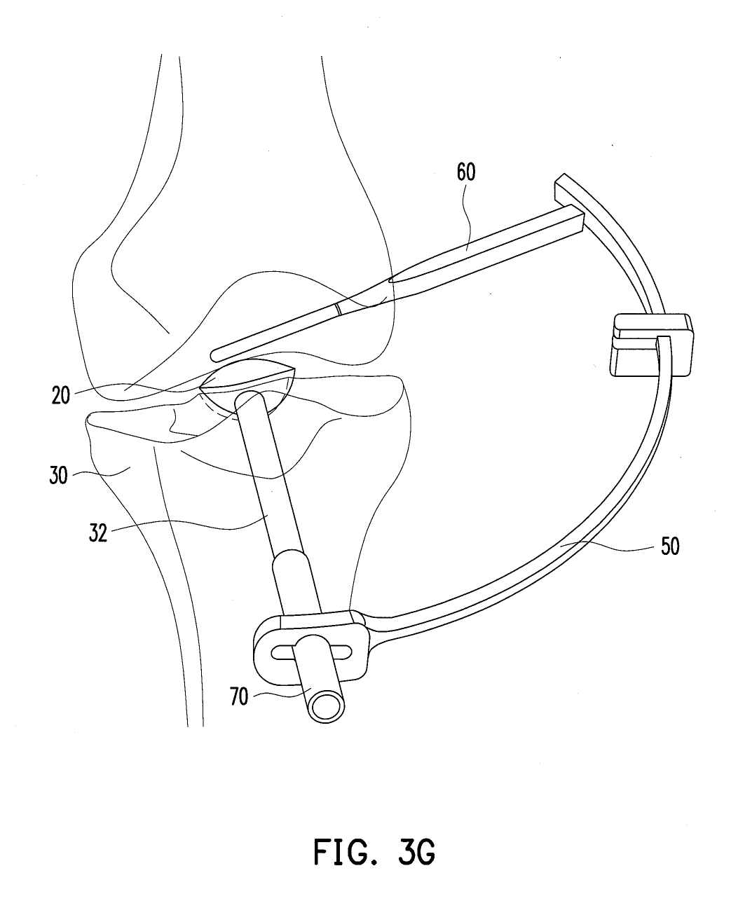

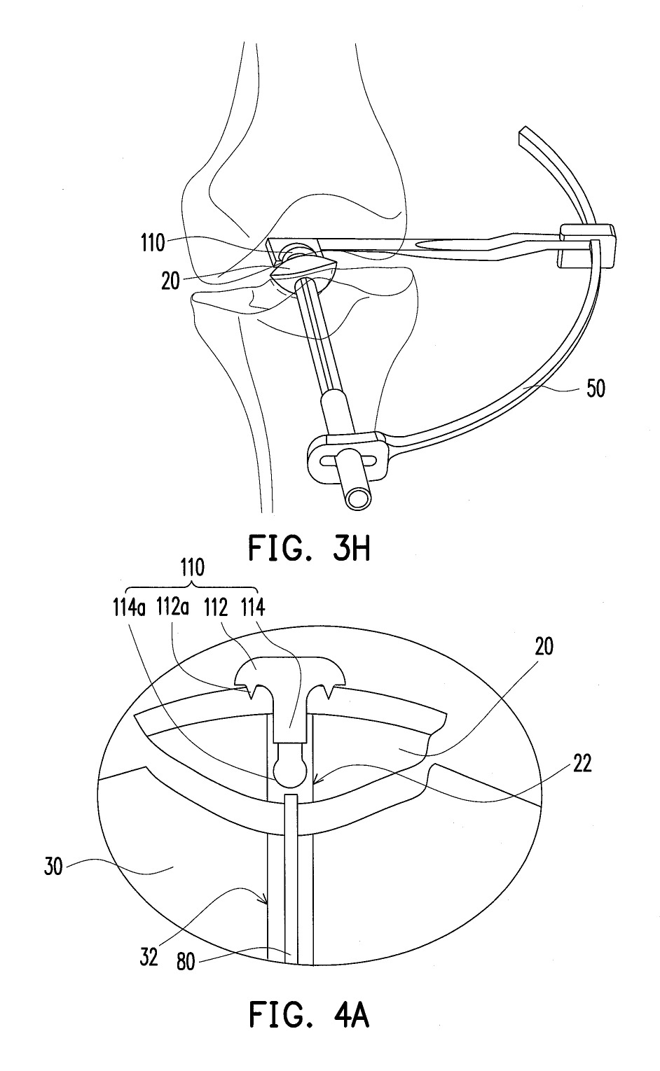

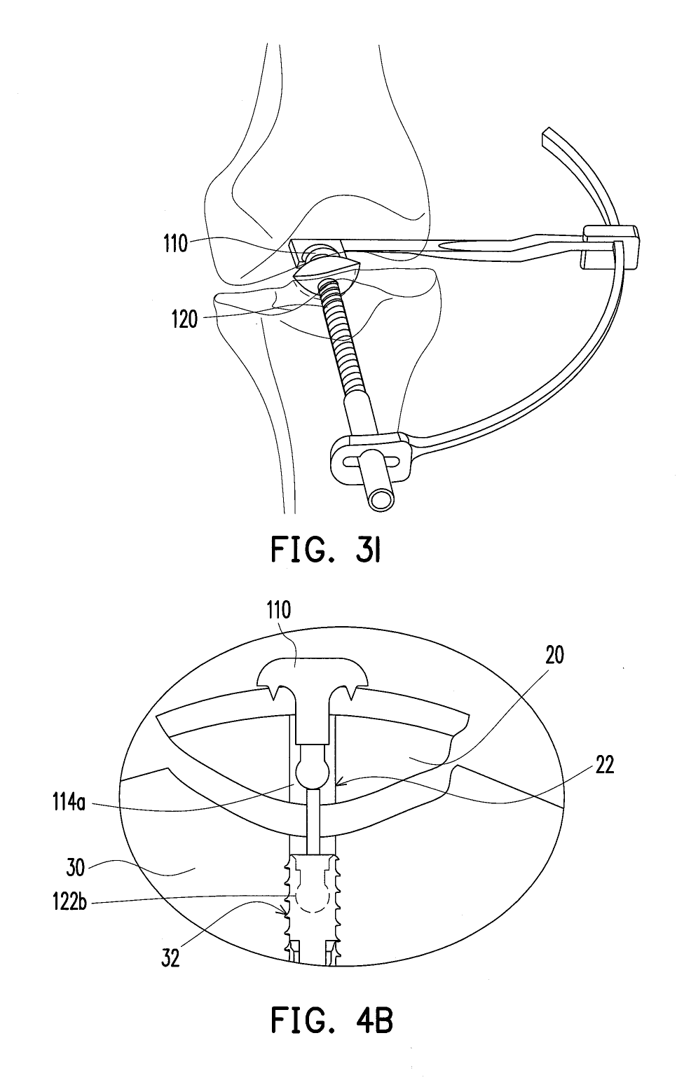

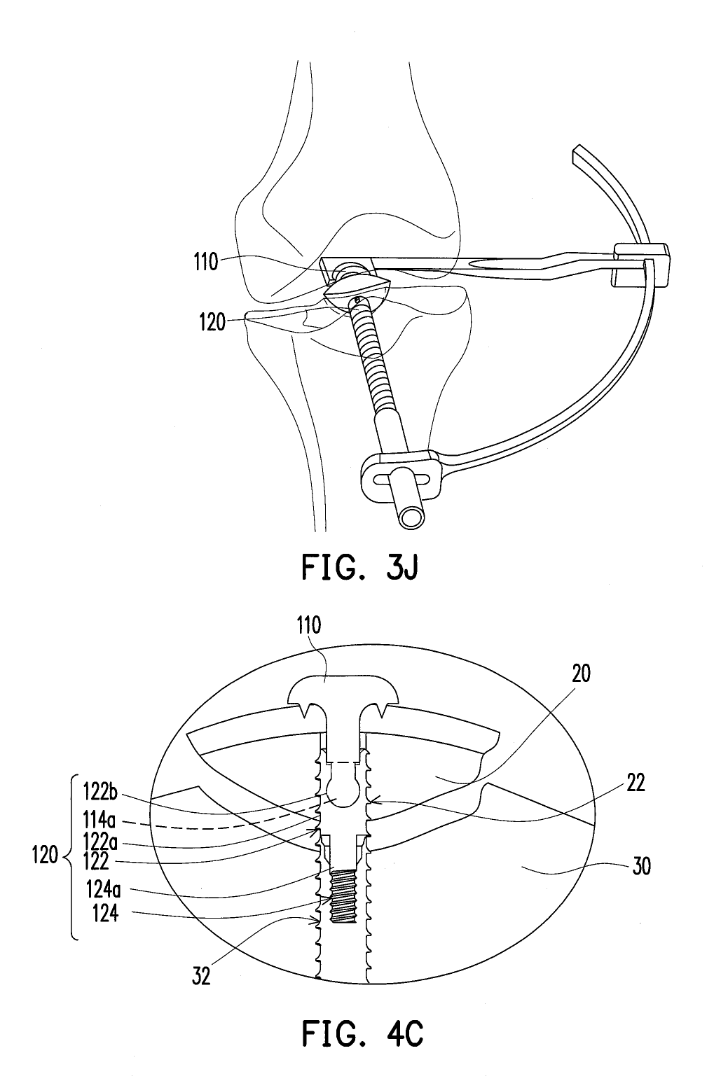

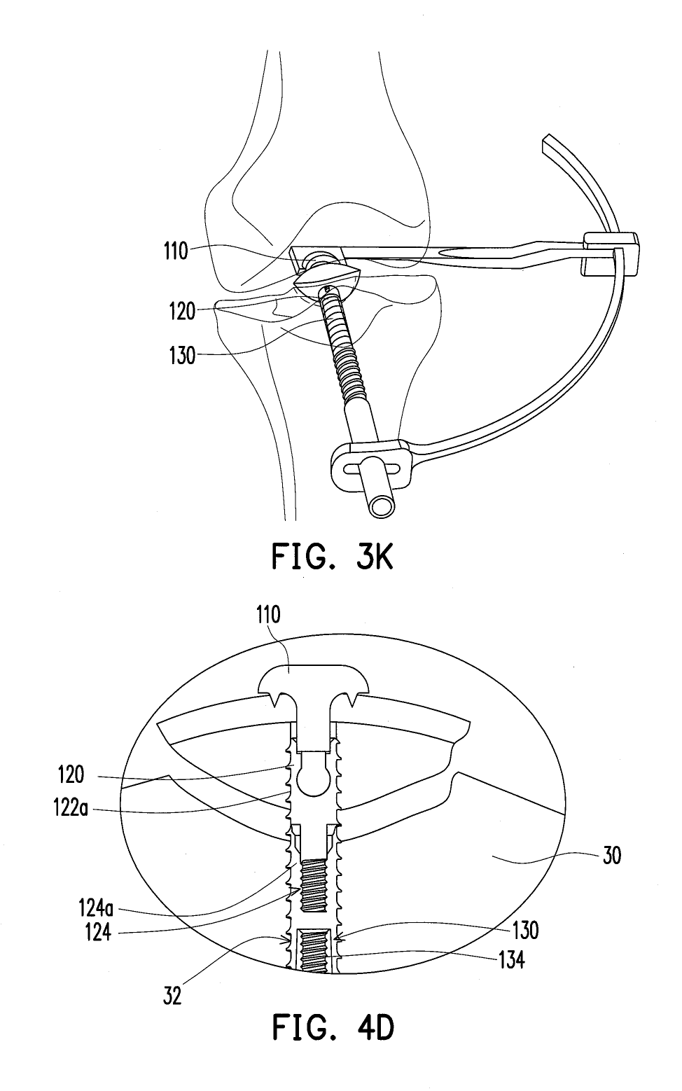

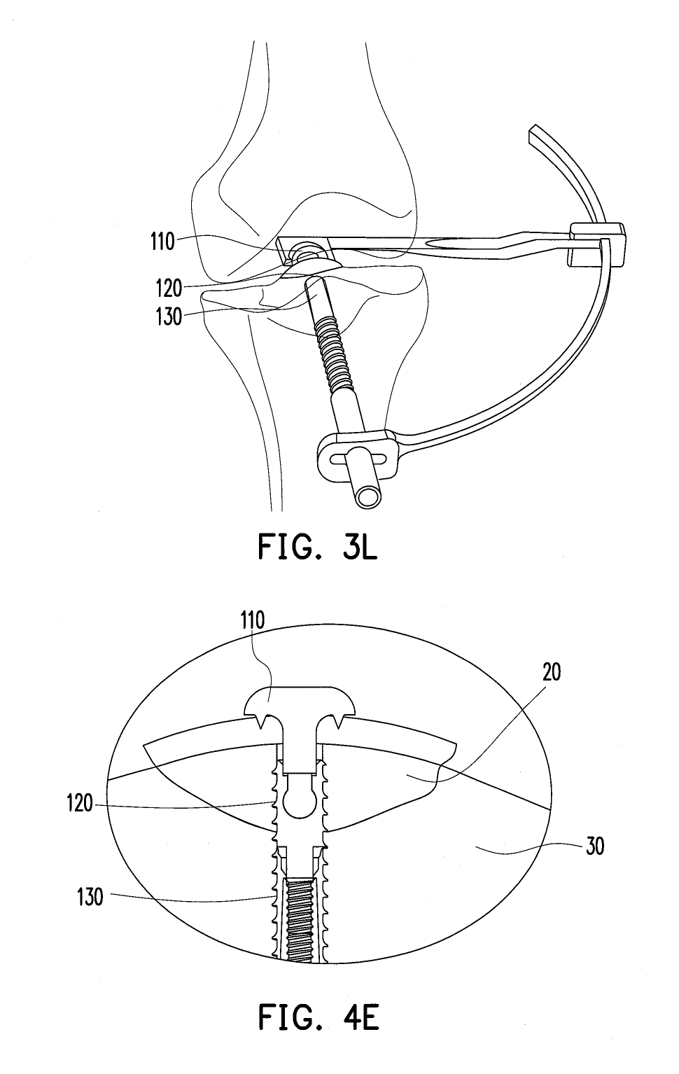

A method for repairing an avulsion fracture is further illustrated in the following to show how to drag the bone fragment toward the bone by using the implant module 100. FIG. 3A through FIG. 3L shows the method for repairing the avulsion fracture according to an embodiment of the disclosure. FIG. 4A through FIG. 4E are partial enlarged views showing the structures in FIG. 3H through FIG. 3L in another view. To the present embodiment, the implant module 100 is implanted by forming openings at a front side and a lateral side of a knee for evading the sciatic nerves and large blood vessels at the rear of the knee, and thereby drags the bone fragment 20 toward the bone 30.

Firstly, referring to FIG. 3A, a locating component 50 is disposed besides the bone 30 and the bone fragment 20. The locating component 50 comprises an arc arm 52, a movable locating hole 54 and a fixed locating hole. The movable locating hole 54 is movably disposed on the arc arm 52, and the fixed locating hole 56 is located at an end of the arc arm 52. In the embodiment, the locating component 50 is disposed at the outer side or the inner side of the knee, and the knee is corresponding to a center portion of the arc arm 52.

Then, referring to FIG. 3B and FIG. 3C, a damper 60 is provided to penetrate the knee from the lateral side of knee, wherein an end of the damper 60 is propped against the bone fragment 20, and the other end of the damper 60 is fixed to the movable locating hole 54 of the locating component 50. In addition, a tube 70 is provided in the fixed locating hole 56 in front of the knee. The damper 60 and the tube 70 are disposed on the arc arm 52 along different normal directions, and thereby an extending direction of the damper 60 intersects an extending direction of the tube 70.

Next, referring to FIG. 3D, a guide pin 80 is provided to penetrate the tube 70, the bone 30 and the bone fragment 20 in sequence, and an end of the guide pin 80 is linked to the damper 80. Herein, the damper 60 is provided with an end propped against the bone fragment 20 and having an opening (not shown) at the end. The guide pin 80 penetrates the opening.

Then, referring to FIG. 3E and FIG. 3F, a hollow drill 90 is provided to penetrate the tube 70 and form a second through hole 32 in the bone 30 and a first through hole 22 in the bone fragment 20 by drilling the knee along the guide pin 80, wherein the second through hole 32 penetrates the bone 30 and connect where the bone fragment 20 tears from the bone 30. And, referring to FIG. 3G, the hollow drill 90 and the damper 60 are removed after the first through hole 22 and the second through hole 32 are formed. Herein, since the first through hole 22 and the second through hole 32 are formed by the same hollow drill 90, the first through hole 22 and the second through hole 32 have the same diameter. However, the first through hole 22 and the second through hole 32 may be formed in different diameters by using different hollow drills due to practical requirements.

The above steps forms the first through hole 22 and the second through hole 32 in the bone fragment 20 and the bone 30 respectively for implanting the implant module 100 into the knee. Referring to FIG. 3H, a cap 110 is disposed at a side of the bone fragment 20 in opposite to the bone fragment 20 by using for example a steel pin to guide the cap 110. As shown in FIG. 4A, the cap 110 comprises a head portion 112 and a stem portion 114 connected to the head portion 112. The head portion 112 comprises a plurality of spikes 112a. The spikes 112a stabs into the bone fragment 20 when the stem portion 114 is inserted into the first through hole 22 and the head portion 112 is propped against the bone fragment 20, to reliably fix cap 110 to the bone fragment 20. The stem portion 114 is provided with first engaging portion 114a at an end far away from the head portion 112.

Next, referring to FIG. 3I and FIG. 3J, the screw 120 is screwed from the front of the knee, through the bone 30 to the bone fragment 20. More specifically, referring to FIG. 4B and FIG. 4C, the screw 120 comprises a first section 122 and a second section 124, wherein an outer diameter of the first section 122 is greater than an outer diameter of the second section 124. The first section 122 has a third external thread 122a on its outer surface. The outer diameter of the first section 122 of the screw 120 is greater than or equal to the diameter of the first through hole 22 and the diameter of the second through hole 32. In the present embodiment, the outer diameter of the first section 122 of the screw 120 is greater than the diameter of the first through hole 22 and the diameter of the second through hole 32, such that the first section 122 of the screw 120 forms internal threads on inner surfaces of the second through hole 32 and the first through hole 22 corresponding to the third external thread 122a when screwing the first section 122 into the first through hole 22 of the bone fragment 20 through the second through hole 32 of the bone 30. The first section 122 is provided with a second engaging portion 122b adapted to be engaged with the first engaging portion 114a of the stem portion 114 when the screw 120 is screwed into the first through hole 22 of the bone fragment 20, and thus the bone fragment 20 is clamped by the cap 110 and the screw 120.

In the another embodiment, the first engaging portion 114a comprises a ball, and the second engaging portion 122b comprises a ball-shaped socket, i.e. the first engaging portion 114a and the second engaging portion 122b are linked by a ball joint. In addition, the second section 124 of the screw 120 has a first external thread 124a on its outer surface. After that, referring to FIG. 2A, FIG. 3K and FIG. 4D, the stud 130 is screwed into the knee from the front of the knee, wherein the stud 130 has a second external thread 132 on its outer surface. The outer diameter of the stud 130 is greater than or equal to the outer diameter of the first section 122 of the screw 120. In the present embodiment, the outer diameter of the stud 130 is approximate to the outer diameter of the first section 122 of the screw 120, and the pitch of the second external thread 132 is smaller than the pitch of the third external thread 122a, such that an internal thread corresponding to the second external thread 132 is formed on the inner surface of the second through hole 32 when the stud 130 is screwed into the second through hole 32. Therefore, the stud 130 can be screwed into the second through hole 32 and fixed to the bone 30 via the second external thread 132.

In other embodiments, the outer diameter of the stud 130 may be equal to that of the first section 122 of the screw 120, and the second external thread 132 is identical to the third external thread 122a. The internal thread on the inner surface of the second through hole 32 formed by the third external thread 122a of the first section 122 of the screw 120 is matched with the second external thread 132 of the stud 130, such that the stud 130 can be directly screwed into the second through hole 32 and fixed to the bone 30. Furthermore, The outer diameter of the stud 130 may also be greater than the outer diameter of the first section 122 of the screw 120, and thereby an internal thread corresponding to the second external thread 132 can be formed on the inner surface of the second through hole 32 when the stud 130 is screwed into the second through hole 32, so as to screw the stud 130 into the second through hole 32 of the bone 30 via the second external thread 132.

In addition, an end of the stud 130 adjacent to the screw 120 is provided with a screw hole 134 for screwing the second section 124 of the screw 120 via the first external thread 124a. The stud 130 is screwed with the screw 120 in the progress toward the rear of the knee as shown in FIG. 3L and FIG. 4E.

Moreover, in the embodiment, the pitch of the second external thread 132 is configured to be smaller than the pitch of the first external thread 124a such that the stud 130 moves with respective to the bone in a distance smaller than a displacement between the screw 120 and the stub 130 when the stud 130 is screwed with the bone 30 via the second external thread 132 and screwed with the first external thread 124a of the screw 120 via the screw hole 134, for dragging the bone fragment toward where the bone fragment 20 tears from the bone 30 and resetting the bone fragment 20 to its original location.

In addition, the screw 120 may be fixed to the bone fragment 20 by various manners such as screwing or adhering, etc., and, in other embodiments, the cap 110 can further be omitted, such that the process of dragging the bone fragment 20 toward where the bone fragment 20 tears from the bone 30 can be accomplished by skipping the steps of screwing the cap 110 and linking the screw 120 and the cap 110.

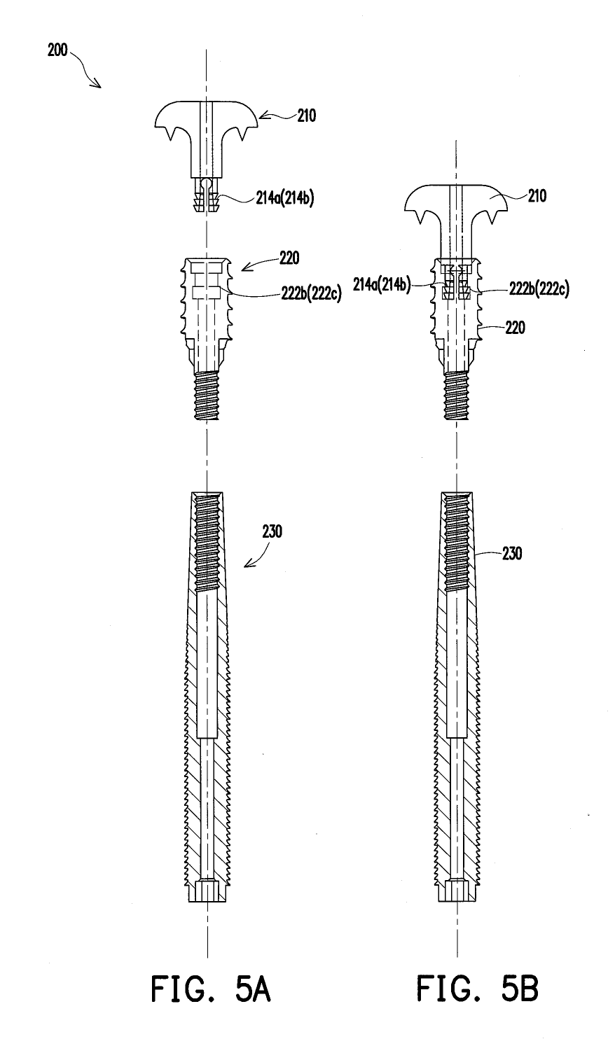

Although the first engaging portion 114a and the second engaging portion 112b are ball joint for linking the cap 110 and the screw 120 together in the above embodiments, other types of the first engaging portion 114a and the second engaging portion 112b may further be proposed. FIG. 5A is an explosive view of an implant module according another embodiment of the disclosure. FIG. 5B shows a cap and a screw of the implant module of FIG. 5A being assembled. Referring to FIG. 5A and FIG. 5B, the implant module 200 comprises a cap 210, a screw 220 and a stud 230. A first engaging portion 214a of the cap 210 comprises a trip 214b, and a second engaging portion 222b of the screw 220 comprises a groove 222c match with the trip 214b. The cap 210 is linked to the screw 220 by engaging the trip 214b with the groove 222c. In other embodiment, the first engaging portion 214a and the second engaging portion 222b are not limited to the above embodiment, and can further be engaged with each other by using other manners such as screwing, adhering, and etc.

In summary, the implant module and the method for repairing the avulsion fracture are provided with a stud having a second external thread in a pitch different from a pitch of a first external thread of a screw, wherein the screw fixed to the bone fragment can be dragged toward the stub in the bone by screwing the stud with the screw, so as to fix the bone fragment to the bone. Furthermore, since the implant module is in a small size, incisions of surgical treatment can be reduced for a short recovery time of patients. In addition, the method for repairing the avulsion fracture is performed by forming openings at the front side and the lateral side of the knee for implanting the implant module, the sciatic nerves and large blood vessels in the rear of the knee are evaded, so as to dramatically reduce time and risks of surgical treatment.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An implant module, for dragging a bone fragment toward where the bone fragment tears from a bone, wherein the bone fragment has a first through hole, and the bone has a second through hole penetrating the bone and being connected to where the bone fragment tears from the bone, the implant module comprising:
a screw, comprising a first section and a second section, wherein the first section is fixed to the bone fragment, and the second section is provided with a first external thread on its outer surface; and
a stud, provided with a second external thread on its outer surface for being screwed with the second through hole of the bone, wherein an end of the stud has a screw hole, and the second section of the screw is adapted to be screwed with the screw hole via the first external thread.

2. The implant module as claimed in claim 1, wherein the first section is provided with a third external thread on its outer surface for being screwed with the first through hole of the bone fragment.

3. The implant module as claimed in claim 1, further comprising:
a cap, for being arranged at a side of the bone fragment and in opposite to where the bone fragment tears from the bone, wherein the cap has a first engaging portion, and the first section of the screw has a second engaging portion adapted to be engaged with the first engaging portion, such that the cap and the screw mutually clamp the bone fragment.

4. The implant module as claimed in claim 3, wherein the cap comprises a head portion and a stem portion connected to the head portion, the stem portion has an end far away from the head portion, and the first engaging portion is located at the end of the stem portion.

5. The implant module as claimed in claim 4, wherein the head portion comprises a plurality of spikes for stabbing into the bone fragment.

6. The implant module as claimed in claim 3, wherein the first engaging portion comprises a ball, and the second engaging portion comprises a ball-shaped socket.

7. The implant module as claimed in claim 3, wherein the first engaging portion comprises a trip, and the second engaging portion comprises a groove.

8. The implant module as claimed in claim 1, wherein an outer diameter of the stud is equal to or greater than an outer diameter of the first section of the screw, and the outer diameter of the first section of the screw is equal to or greater than a diameter of the first through hole and the diameter of the second through hole.

9. The implant module as claimed in claim 1, wherein a pitch of the second external thread is smaller than a pitch of the first external thread.

10. A method for repairing an avulsion fracture, for dragging a bone fragment toward where the bone fragment tears from a bone, the method comprising:
forming a first through hole in the bone fragment, and forming a second through hole in the bone, wherein the second through hole penetrates the bone and is connected to where the bone fragment tears from the bone;
screwing a screw to the bone fragment from a side adjacent to where the bone fragment tears from the bone, wherein a first section of the screw is fixed to the bone fragment; and
screwing a stud to the bone from a side far away from where the bone fragment tears from the bone, wherein the stud with a second external thread on an outer surface thereof is screwed with the second through hole of the bone, and an end of the stud has a screw hole screwed with a first external thread on a second section of the screw, to drag the bone fragment toward where the bone fragment tears from the bone.

11. The method as claimed in claim 10, wherein the screw is fixed to the bone fragment by screwing a third external thread on an outer surface of the first section with the first through hole.

12. The method as claimed in claim 10, further comprising:
providing a cap at a side of the bone fragment in opposite to where the bone fragment tears from the bone, wherein the cap has a first engaging portion inserted into the first through hole, and the first section of the screw has a second engaging portion engaged with the first engaging portion, such that the cap and the screw mutually clamp the bone fragment.

13. The method as claimed in claim 12, wherein the cap comprises a head portion and a stem portion connected to the head portion, the stem portion has an end far away from the head portion, and the first engaging portion is located at the end of the stem portion.

14. The method as claimed in claim 13, wherein the head portion comprises a plurality of spikes for stabbing into the bone fragment.

15. The method as claimed in claim 12, wherein the first engaging portion comprises a ball, and the second engaging portion comprises a ball-shaped socket.

16. The method as claimed in claim 12, wherein the first engaging portion comprises a trip, and the second engaging portion comprises a groove.

17. The method as claimed in claim 10, wherein an outer diameter of the stud is equal to or greater than an outer diameter of the first section of the screw, and the outer diameter of the first section of the screw is equal to or greater than a diameter of the first through hole and the diameter of the second through hole.

18. The method as claimed in claim 10, wherein a pitch of the second external thread is smaller than a pitch of the first external thread.

19. The method as claimed in claim 10, wherein the first through hole and the second through hole are formed by:
disposing a locating component between the bone and the bone fragment, wherein the locating component comprises an arc arm, a movable locating hole movably configured on the arc arm, and a fixed locating hole located at an end of the arc arm;
providing a damper located in the movable locating hole and propped against the bone fragment;
providing a tube in the fixed locating hole;
providing a guide pin to penetrate the tube, the second through hole and the first through hole in sequence, wherein an end of the guide pin is linked to the damper; and
forming the second through hole and the first through hole respectively in the bone and the bone fragment along the guide pin by using a hollow drill.

20. An implant module, comprising:
a screw, comprising a first section and a second section, wherein the second section is provided with a first external thread on its outer surface; and
a stud, provided with a second external thread on its outer surface, wherein an end of the stud has a screw hole, and the second section of the screw is adapted to be screwed with the screw hole via the first external thread.

21. The implant module as claimed in claim 20, wherein the first section is provided with a third external thread on its outer surface.

22. The implant module as claimed in claim 20, further comprising:
a cap having a first engaging portion, wherein the first section of the screw has a second engaging portion adapted to be engaged with the first engaging portion.

23. The implant module as claimed in claim 22, wherein the cap comprises a head portion and a stem portion connected to the head portion, the stem portion has an end far away from the head portion, and the first engaging portion is located at the end of the stem portion.

24. The implant module as claimed in claim 23, wherein the head portion comprises a plurality of spikes.

25. The implant module as claimed in claim 22, wherein the first engaging portion comprises a ball, and the second engaging portion comprises a ball-shaped socket.

26. The implant module as claimed in claim 22, wherein the first engaging portion comprises a trip, and the second engaging portion comprises a groove.

27. The implant module as claimed in claim 20, wherein an outer diameter of the stud is equal to or greater than an outer diameter of the first section of the screw.

28. The implant module as claimed in claim 20, wherein a pitch of the second external thread is smaller than a pitch of the first external thread.
